(19) United States
(12) Patent Application Publication
(10) Pub. No.: US 2008/0102515 A1
(43) Pub. Date: May 1, 2008

(54) USE OF MINING WASTE AND CONCENTRATES CONTAINING PYRITE, IN THE CULTURE OF IRON-OXIDIZING AND SULFUR-OXIDIZING MICROORGANISMS AS AN ENERGY SOURCE FOR BACTERIAL GROWTH

(75) Inventors: Pedro Antonio Morales Cerda, La Reina (CL); Leandro Mauricio Padilla Iglesias, Providencia (CL); Ricardo Badilla Ohlbaum, La Reina (CL)

Correspondence Address: MERCHANT & GOULD PC, P.O. BOX 2903, MINNEAPOLIS, MN, 55402-0903, US

(73) Assignee: BIOSIGMA S.A., Colina (CL)

(21) Appl. No.: 11/862,043

(22) Filed: Sep. 26, 2007

(30) Foreign Application Priority Data
Oct. 27, 2006 (CL) 2910-2006

(51) Int. Cl. C12N 1/20

(52) U.S. Cl. 435/262

(57) ABSTRACT

The invention publishes the use of mining products and sub-products that contain pyrite, such as copper concentrates, and waste from the process in which these concentrates are obtained, known as scavenger tail, as an energy source for the large-scale culture of an association of microorganisms that are useful for ore bioleaching, and that includes both isolated microorganisms, and native microorganisms from the worked ores. In particular, the invention publishes the use of mining waste known as scavenger tail from the flotation process, in the culture of an association of isolated microorganisms of the Acidithiobacillus ferrooxidans y Acidithiobacillus thiooxidans type together, with or without other native microorganisms from the worked ores.

### BACKGROUND OF THE INVENTION

The invention publishes the use of pyrite-containing mining products and sub-products, such as for example, copper concentrates, and the waste from the process in which these concentrates are obtained, known as “scavenger tail”, as an energy source for the large-scale culture of an association of microorganisms that are useful in ore bioleaching, and which includes both isolated micro-organisms, and native microorganisms present in the worked ore. In particular, the invention publishes the use of mining waste, known as “scavenger tail”, from the flotation process, in the culture of an association of isolated microorganisms of the Acidithiobacillus ferrooxidans and Acidithiobacillus thiooxidans type together, with or without other native microorganisms of the worked ores.

### SUMMARY OF THE INVENTION

Typically, in microorganism culture, artificial or expressly prepared culture mediums are used, frequently starting from highly pure organic and/or inorganic chemical products. This normally has the purpose of controlling to a maximum the variables related to the requirements of the microorganisms, and avoiding all potential sources of contamination or inhibition of microbial growth.

For example, the laboratory-scale growth of At. ferrooxidans and At. thiooxidans has been described by Silverman, M. P. & Lundgren D. G. 1959. “Studies on the chemoautotrophic iron bacterium ferrobacillus ferroooxidans I. An Improved Medium and a Harvesting Procedure for Securing High Cell Yields” Journal of Bacteriology. 77: 642-647, and Cook, T. M. 1964. “Growth of Thiobacillus thiooxidans in shaken culture” Journal of Bacteriology. 88: 620-623.

The previous approach is highly appropriate for the culture of microorganisms at a laboratory level, and even sometimes at a pilot-test level. However, due to economic considerations it could become impractical, above all if it deals with the large-scale production of biomass. This problem is normally solved by using reagents of a technical-industrial grade, which decreases the cost of the medium, but increases potential sources of contamination, besides adding impurities that could inhibit microorganism growth.

Thus, for culturing microorganisms in industrial conditions, formulations based on ammonium sulfate and potassium phosphate of a technical level (Hackl et al. U.S. Pat. No. 5,089,412) have been described. Likewise, in the applications for Chilean patents CL 2731-2004, and CL 2101-2005, the culture mediums known as 9K modified (3.0 g/L of (NH₄)₂SO₄, 0.5 g/L of K₂HPO₄, 0.5 g/L of MgSO₄.7H₂O, 0.1 g/L of KCl and 0.1 g/L of Ca(NO₃)₂, 30 g/L of FeSO₄.7H₂O) and 9KS (3.0 g/L of (NH₄)₂SO₄, 0.5 g/L of K₂HPO₄, 0.5 g/L of MgSO₄.7H₂O, 0.1 g/L of KCl, 0.1 g/L of Ca(NO₃)₂, 1% of elementary sulfur or another reduced sulfur compound, are respectively used.

It is a known fact that in the cultivation of microorganisms in mediums such as those described, the final biomass concentration is limited by the concentration of the substrate used as an energy source and by the inhibition of growth exercised both by the substrate and the products of its metabolism generated during microbial growth [LaCombe, J., Lueking, D. 1990. “Growth and maintenance of Thiobacillus ferrooxidans cells” Applied and Environmental Microbiology. 56: 2801-2806; Nagpal, S. 1997. “A structured model for Thiobacillus ferrooxidans growth on ferrous Iron” Biotechnology and Bioengineering. 53. 310-319].

On the other hand, the type of microorganism obtained depends on the type of energy source used, iron in the form of Fe²⁺ compounds for iron-oxidizing micro-organisms, and sulfur compounds—in a −2, 0 y+4 state of oxidation—for sulfur-oxidizing microorganisms. The above constitutes a limiting factor for the design of a mixed biomass (iron and sulfur oxidizing) production process, because it imposes different production conditions—substrates and pH—for each strain.

In the event of wanting to cultivate two or more microorganism species, it seems attractive to use the same culture medium, or even culture the microorganisms together. In this way, the number of stages in the process is decreased, the complexity of the operation is simplified, and in some cases, it is possible to take advantage of the inherent characteristics of the underlying biochemistry.

Iron sulfates, such as pyrite (FeS₂) or the materials that contain it, are sources of reduced iron and sulfur and therefore constitute an interesting alternative for the production of mixed leaching biomass.

Schippers, A., Jozsa, P. G., Sand, W. 1996. “Sulfur chemistry in bacterial leaching of pyrite”. Applied and Environmental Microbiology. 62: 3424-3431, propose the formation of thiosulfate (S₂O₃²⁻) during the pyrite degradation cycle. This compound can follow a series of abiotic reactions, or be used as an energy source by sulfur-oxidizing bacteria, which gives occasion to propose the joint culture of iron-oxidizing and thio-oxidizing microorganisms on materials containing pyrite.

Finally, regarding the use of pyrite, or materials that contain it, the existing studies propose different approaches, for example, patents WO0136693, WO0071763 and WO2004027100 propose its use as a source of sulfuric acid. In document WO0136693 the use of pyrite is associated to leaching systems in which sulfuric acid is not added; in document WO0071763 its use is associated with the replenishment of acid when the ore shows a high demand for it; and in document WO2004027100 they are used to replace part of the necessary acid. In other documents such as U.S. Pat. No. 6,110,253 and application US2005103162, pyrite is used as a mechanism to increase the temperature in the heap, as when it is bio-oxidized, it generates heat, which according to the previously-mentioned text, makes it possible to practice bioleaching with thermophile microorganisms.

Other uses of pyrite are found for example, in the works of de Bacelar-Nicolau, P. & Jonson, B. 1999. “Leaching of pyrite by acidophilic heterotrophic iron-oxidizing bacteria in pure and mixed cultures”. Applied and Environmental Microbiology. 65: 585-590, and Chong, N., Karamanev, D. G., Margaritas, A. 2002. “Effect of particle-particle shearing on the bioleaching of sulfide minerals”. Biotechnology and Bioengineering. 80: 349-357, in which the growth of micro-organisms such as At. ferrooxidans on pyrite as a source of energy is shown at laboratory scale. Nevertheless, the rate of growth in this material appears to be affected by friction between the solid particles.

Therefore, as far as we know, there is still a lack of lower-priced culture mediums to enable the feasible large-scale production of microorganisms useful in bioleaching; and we don't know of processes in which pyrite is effectively used as an energy source for biomass growth either.

### INVENTION SPECIFICATIONS

For a better understanding of the processes, the following should be understood as:
a) ATCC: “American Type Culture Collection”,
b) Ore bioleaching in troughs: a process that is carried out in a tank with a false bottom where the ore is loaded and flooded with the leaching solution which is made to circulate through the ore particles in the presence of acidophilic microorganisms, and the copper is extracted dissolved in an acid solution.
c) Bioleaching of ores in dumps: ores below the cut-off grade, which are extracted from an open-pit mine, are stored as run of mine or after primary crushing, in gorges that have the appropriate characteristics to control the infiltration of solutions or on surfaces where a waterproof sheet has been previously installed. The surface is irrigated with leaching solution, in the presence of acidophilic microorganisms, and the copper dissolved in an acid solution is extracted from the base.
d) Ore heap bioleaching: In this process, the ore that has been crushed down to a specific grading is collected on a water-proof surface with a slight slope, and the leaching solution is irrigated over the surface in the presence of acidophilic microorganisms, and the copper dissolved in an acid solution is extracted from the base.
e) Bioleaching of on-site ore: deposits of ore in their natural state or that have been broken up during previous mining operations are directly leached on-site, irrigating the surface with leaching solution, in the presence of acidophilic microorganisms, and the copper dissolved in an acid solution is extracted from the base.
f) Ore bioleaching in stirred tanks or reactors: the bioleaching process takes place in a mechanically stirred tank where the finely divided ore is mixed with the leaching solution, forming a slurry with a solid content of up to 20%, with the presence of acidophilic microorganisms, extracting the copper dissolved in an acid solution.
g) Bioleaching of tailing ponds: tailings that originate in the flotation process and contain lower quantities of the metal present in the ore are stored in dams, from where they are then extracted for leaching, either in heaps or by stirring, in the presence of acidophilic microorganisms, and the copper is extracted dissolved in an acid solution.
h) Biomass: mass of live organisms produced in a specific area or volume.
i) Scavenger Tail: Sand resulting from a flotation cell circuit of the sand from the main ore cleaning circuit.
j) DSM: “Deutsche Sammlung von Mikroorganismen und Zellkulturen GmbH” German Type Microorganism Culture.
k) Inoculum: pure or mixed bacterial culture which will act as active biological material during the bioleaching process.
l) Passivation: decrease in ore leaching speed as a consequence of the accumulation of layers of sulfur and poli-sulfurs on its surface.
m) PLS: aqueous solution generated during the bioleaching process that contains the metallic ions that have been leached from the ore. This solution constitutes the feed for the PLS solvent extraction plant.
n) Raffinate: copper-depleted aqueous solution, resulting from the process of copper solvent extraction.
o) Mixed energy source: substrate that allows simultaneous growth of iron and sulfur oxidizing microorganisms.
p) Mixed biomass: mass of microorganisms capable of oxidizing reduced iron and sulfur compounds.

In order to produce isolated microorganisms on a large scale, that will be useful for the bioleaching of sulfide metal ores, a process has been developed that is based on the use of bioreactors, in which it is possible to lower the costs of culture mediums in order to grow these microorganisms. This cost reduction is based on the use of concentrates, or of pyrite-containing waste ore from the ore flotation process, such as for example, the waste known as scavenger tail, to partly replace the standard culture medium, as an energy source for two different types of microorganisms that grow together: Acidithiobacillus ferrooxidans and Acidithiobacillus thiooxidans.

This process also furnishes advantages regarding the quantity of microorganisms, their adaptation to the solid phase, and furnishes advantages related to copper recovery and to the obtaining of iron in an oxidized state +3 as well.

Of the waste products that can be used, the scavenger tail from the flotation process has a typical composition which is shown in Table 1. This composition contains a not inconsiderable amount of pyrite of approximately 20%, which may be used advantageously for the joint cultivation of the above-mentioned microorganisms.

TABLE 1
Mineralogical composition of scavenger tail, taking into account 100%
of opaque ore

| | % | | | | | | |
|---|---|---|---|---|---|---|---|
| Ore | Weight | % Vol. | % S | % Cu | % Fe | % Mo | % Zn |
| Chalcopyrite | 2.08 | 1.51 | 0.73 | 0.72 | 0.63 | | |
| Chalcosite | 0.53 | 0.29 | 0.11 | 0.43 | | | |
| Covellite | 0.62 | 0.41 | 0.21 | 0.41 | | | |
| Bornite | 1.37 | 0.82 | 0.35 | 0.86 | 0.15 | | |
| Pyrite | 19.28 | 11.76 | 10.30 | | 8.98 | | |
| Molybdenite | 0.94 | 0.61 | 0.38 | | | 0.57 | |
| Sphalerite | 0.05 | 0.04 | 0.02 | | | | 0.04 |
| Magnetite | 0.21 | 0.12 | | | 0.15 | | |
| Limonite | 0.20 | 0.16 | | | 0.13 | | |
| Rutile | 0.17 | 0.12 | | | | | |
| Gangue | 74.54 | 84.16 | | | | | |
| Total | 100.00 | 100.00 | 12.08 | 2.42 | 10.05 | 0.57 | 0.04 |

As it has been known for a long time, pyrite can be used as an energy source by Acidithiobacillus ferrooxidans type microorganisms, the activity of which may be represented by the following formula:

FeS₂+6Fe³⁺+3H₂O→7Fe²⁺+S₂O₃²⁻+6H⁺

7Fe²⁺+7/4O₂₊₇H⁺→7Fe³⁺+7/2H₂O+At. ferrooxidans

FeS₂+7/4O₂+H⁺→Fe³⁺+S₂O₃²⁻+½H₂O+At. ferrooxidans reaction (i)

As observed in reaction (1), one of the products is thiosulfate, which contemplates sulfur in an intermediate state of oxidation, and which is useful as an energy source for microorganisms of the Acidithiobacillus thiooxidans type, in accordance with the following reaction:

S₂O₃²⁻+H₂O+2O₂→2SO₄²⁻+2H⁺+At. thiooxidans reaction (ii)

Therefore, simultaneous cultivation of Acidithiobacillus ferrooxidans type and Acidithiobacillus thiooxidans type microorganisms, together with other micro-organisms, takes advantage of the presence and formation of species that can be used as an energy source, iron (II) and thiosulfate, respectively.

Taking into consideration that part of the conventional culture medium has not been replaced by a waste product such as scavenger tail that costs nothing, it appears obvious that this culture is less expensive than the culture that uses conventional medium. Furthermore, as two microorganisms are cultivated simultaneously, there are further decreases in costs associated with facilities, reactors, control systems, etc. that would otherwise have to be doubled.

In addition, joint cultivation using scavenger tail waste makes it possible to obtain a higher concentration of microorganisms than what is normally obtained when the same microorganisms are cultivated separately. This has economic significance, which may be evaluated by the reduction of the equipment needed to achieve a given target concentration when new facilities are being planned, or by a higher production capacity in currently operating facilities.

Based on the studies carried out presented below in the examples, it is possible to affirm that the association of microorganisms that includes isolated microorganisms mixed with native microorganisms from the ore, grows normally in the medium modified with scavenger tail waste. The above constitutes progress in regard to the state of the art, as it lowers culture costs by reducing the costs of the culture medium.

Furthermore, in accordance with the reactions set forth previously, a higher concentration of the Acidithiobacillus thiooxidans species will naturally be produced, or likewise, a higher relative growth of the Acidithiobacillus thiooxidans species will naturally also be produced. This may or may not be an advantage, depending on considerations regarding subsequent processes in which the generated biomass is used. Nevertheless, if it is desired or necessary, microorganism growth may be balanced by incorporating Fe⁺² as ferrous sulfate (FeSO₄.7H₂O).

As indicated, in practice the invention is verified by replacing part of the standard microorganism culture medium of the microorganisms by waste that contains pyrite, such as scavenger tail from the ore flotation process. The fraction of the culture medium that is replaced is the one that corresponds to iron and sulfur species, and it is possible to replace a sizeable part of it. For example in a cultivation medium modified in accordance with the invention, 1 to 100 g/L of scavenger tail may be used.

Furthermore, and because waste such as scavenger tail contains solids, the microorganisms are able to adapt to solid phase sulfur oxidizing. This adaptation is useful, and also represents a technical step forward, because, as the microorganisms are adapted to the solid phase, they will rapidly populate the materials stationed in heaps, dumps, tailing dams or other on-site operations in which they are used, decreasing the time associated to their leaching.

In addition, copper in pyrite-containing waste, particularly in scavenger tail, where copper constitutes almost 2.5%, shall be released into the solution, remaining free to be recovered by means of the usual copper recovery processes, and therefore increasing the general productivity of the process. Once again, this means progress, seeing as in state-of-the-art processes the copper found in this waste is lost.

Finally, and in accordance with the reactions presented previously, iron enrichment to a state of oxidation +3 is produced in the cultivation medium. As technically known, the presence of Fe³⁺ promotes the leaching of secondary ores, and therefore this also represents an advantage over other processes.

### BRIEF DESCRIPTION OF THE DRAWINGS

FIG. 1: This figure presents the batch mode growth curve of an association of microorganisms in a culture medium modified by the incorporation of scavenger tail, as described in Example 1.

FIG. 2: This figure presents the contents of At. ferrooxidans Wenelen DSM 16786 (black bars) and At. thiooxidans Licanantay DSM 17318 (white bars) in a biomass propagation bioreactor operated in continuous mode, using culture medium modified by the incorporation of scavenger tail, as described in Example 2.

### DETAILED DESCRIPTION OF THE PREFERRED EMBODIMENT

### Example 1

In order to determine the growth kinetics and biomass yield of the association of Wenelen DSM 16786 and Licanantay DSM 17318 microorganisms, using medium modified by the incorporation of scavenger tail, an experiment is carried out using the following protocol:

### Protocol

Bacterial growth took place in a 6 m³ useful volume reactor.

The culture medium used to propagate the microorganisms was prepared by suspending scavenger tail (at a 1.25% pulp density) in a nutrient solution composed as follows: 75 g FeSO₄/L, 0.99 g (NH₄)₂SO₄/L, 0.128 g NaH₂PO₄.H₂O/L, 0.0525 g KH₂PO₄/L, 0.1 g MgSO₄.7H₂O/L, 0.021 g CaCl₂/L. The pH of the cultivation medium was adjusted to 1.8.

To start the culture, 5,400 L of cultivation medium were mixed with 600 L of bacterial inoculum carrying Wenelen DSM 16786 and Licanantay DSM 17318 microorganisms.

To enable growth of the microorganisms in the reactor, air enriched with 0.5% of CO₂ was supplied. The temperature of the reactor was controlled at 30° C. The pH in the reactor was controlled by adding H₂SO₄

The reactor was operated in batch mode for 15 days. During reactor operation, microorganism growth was monitored by microscopic count, using a Petroff-Hausser chamber.

### Results

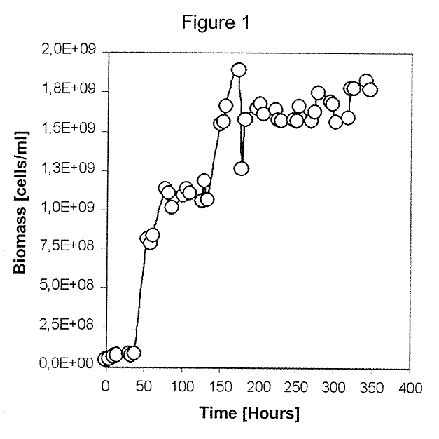

As observed in FIG. 1, the concentration of microorganisms in the culture medium modified with scavenger tail rapidly increased, attaining a maximum concentration of microorganisms of 1.7×10⁹ cells/ml in 6 days. Based on the data obtained during the exponential growth period, it was possible to determine a specific growth speed of 0.069 h⁻¹.

### Example 2

In order to prove that the association of Wenelen DSM 16786 and Licanantay DSM 17318 microorganisms can effectively be propagated continuously using a medium modified by the incorporation of scavenger tail, an experiment is carried out using the following protocol.

### Protocol

Bacterial growth took place in a 50 m³ industrial reactor.

The culture medium used in the propagation of the microorganisms was prepared suspending scavenger tail (at a 0.125% slurry density) in a nutrient solution composed as follows: 8 g FeSO₄/L, 0.99 g (NH₄)₂SO₄/L, 0.128 g NaH₂PO₄.H₂O/L, 0.0525 g KH₂PO₄/L, 0.1 g MgSO₄.7H₂O/L, 0.021 g CaCl₂/L. The pH of the cultivation medium was adjusted to 1.8.

To start the culture, 44 m³ of culture medium were mixed with 6 m³ of bacterial inoculum, carrying Wenelen DSM 16786 and Licanantay DSM 17318 microorganisms.

To allow the growth of the microorganisms in the reactor, air enriched with 0.5% of CO₂ was supplied. The temperature of the reactor was controlled at 300. The pH in the reactor was controlled by adding H₂SO₄.

During operation of the reactor, the growth of the microorganisms was monitored by microscopic count, using a Petroff-Hausser chamber.

Characterization of the microorganisms present in the reactor was carried out using the quantitative PCR (qPCR) technique.

The reactor was operated in batch mode for 7 days, after which the reactor was operated continuously, by feeding the culture medium of the indicated composition at a rate of 360 L/h.

During the continuous operation phase of the reactor, samples were taken for its characterization by qPCR.

### Results

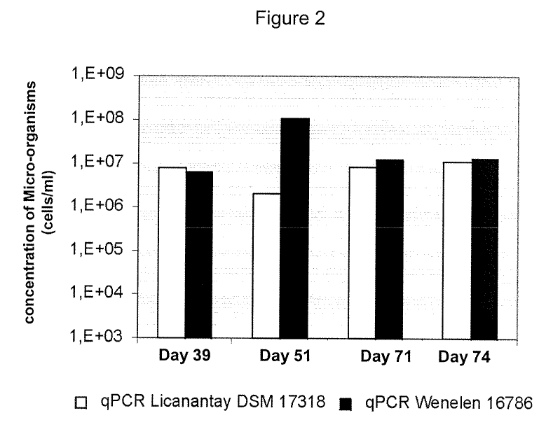

As shown in FIG. 2, continuous operation of a bioreactor using a medium modified by the incorporation of scavenger tail, effectively allows propagation of the At. ferrooxidans and At. thiooxidans microorganism species.

### ADVANTAGES OF THIS INVENTION

In order to evaluate the lower costs of the cultivation medium as a result of the incorporation of scavenger tail, a 2,000-ton heap is contemplated, irrigated with a flow of 480 L/h during 365 days, with continuous inoculation at a concentration of 1×10⁸ cells/mL.

The indicated conditions determine the need to produce microorganisms at 360 L/h at a concentration of 1.3×10⁸ cells/ml. If a value of US$350.—per ton of ferrous sulfate is considered, at a concentration of 8 g/l of ferrous sulfate, the total substitution of this reagent by scavenger tail would produce savings of 8,830 dollars per year, as scavenger tail costs nothing. Typical copper mining operations involve bioleaching of over 2 million tons of ore per year (for example, the Cerro Colorado operation in Chile) and therefore, savings associated to the use of pyrite instead of ferrous sulfate and a separate source of sulfur is of more than US$8 million per year, making continuous inoculation of bacteria to the process sustainable.

In the event that copper concentrates containing pyrite are used as an energy source for bacterial growth, the fraction of bioleached copper is incorporated to the bioleaching solution together with the microorganisms, whereas the copper that remains in the concentrate can be sent to the smelter in the shape of higher grade copper concentrate, as a major part of the pyrite is eliminated during the bacterial growth process.

1. A method using mining waste and concentrates containing pyrite, wherein they are used in the culture of iron-oxidizing and sulfur-oxidizing microorganisms as a source of energy for bacterial growth in the process in which ores are bioleached in reactors.

2. The method of using mining waste and concentrates containing pyrite, in accordance with claim 1, wherein the waste that contains pyrite, used in the cultivation of microorganisms is scavenger tail, which corresponds to sand resulting from a flotation cell ore cleaning circuit.

3. The method of using mining waste and concentrates containing pyrite, in accordance with claim 1, wherein the iron-oxidizing and sulfur-oxidizing microorganisms are mixtures of isolated microorganisms with native microorganisms from the ore that is worked.

4. The method of using mining waste and concentrates containing pyrite, in accordance with claim 3, wherein the isolated micro-organisms used are Wenelen DSM 16786 and Licanantay DSM 17318.
